(19) United States
(12) Patent Application Publication
(10) Pub. No.: US 2010/0262020 A1
(43) Pub. Date: Oct. 14, 2010

(54) PROBE APPARATUS FOR RECOGNIZING ABNORMAL TISSUE

(75) Inventors: Vadim Backman, Chicago, IL (US); Bradley Gould, Evanston, IL (US); Andrew Cittadine, Chicago, IL (US); Jeremy Rogers, Chicago, IL (US); Hemant Roy, Highland Park, IL (US)

Correspondence Address: PILLSBURY WINTHROP SHAW PITTMAN LLP, P.O. BOX 10500, MCLEAN, VA, 22102, US

(73) Assignees: American Biooptics LLC, Evanston, IL (US); Northshore University Healthsystem, Evanston, IL (US); Northwestern University, Evanston, IL (US)

(21) Appl. No.: 12/684,837

(22) Filed: Jan. 8, 2010

Related U.S. Application Data
(60) Provisional application No. 61/143,407, filed on Jan. 8, 2009.

(51) Int. Cl. A61B 6/00

(52) U.S. Cl. 600/478

(57) ABSTRACT

The present invention relates to probe apparatuses and component combinations thereof that are used to recognize possibly abnormal living tissue using a detected early increase in microvascular blood supply and corresponding applications. In one embodiment there is disclosed an apparatus that emits broadband light obtained from a light source onto microvasculature of tissue disposed within a human body and receives interacted light that is obtained from interaction of the broadband light with the microvasculature for transmission to a receiver. Different further embodiments include combinations of optical fibers, polarizers and lenses that assist in the selection of a predetermined depth profile of interacted light. In another embodiment, a kit apparatus is described that has various probe tips and/or light transmission elements that provide for various combinations of predetermined depth profiles of interacted light. In a further embodiment, a method of making a spectral data probe for depth range detection selectivity for detection of blood within microvasculature of tissue is described.

### RELATED APPLICATIONS

This application claims priority to U.S. Provisional Patent Application No. 61/143,407 filed Jan. 8, 2009 entitled “Probe Apparatus for Recognizing Abnormal Tissue”, the entire contents of which is incorporated by reference herein.

This application is related to co-pending U.S. patent application Ser. No. 11/604,653 filed Nov. 27, 2006, entitled “Method of Recognizing Abnormal Tissue Using the Detection of Early Increase in Microvascular Blood Content”, the disclosure of which is incorporated in its entirety by reference, which application claims priority to U.S. Application No. 60/801,947 entitled “Guide-To-Colonoscopy By Optical Detection Of Colonic Micro-Circulation And Applications Of Same”, which was filed on May 19, 2006, the contents of which are expressly incorporated by reference herein.

This application is also related to co-pending U.S. patent application Ser. No. 11/604,659 filed Nov. 27, 2006 and entitled “Apparatus For Recognizing Abnormal Tissue Using The Detection Of Early Increase In Microvascular Blood Content,” the contents of which are expressly incorporated by reference herein.

This application is also related to co-pending U.S. patent application Ser. No. 11/261,452 entitled “Multi-Dimensional Elastic Light Scattering”, filed Oct. 27, 2005, the contents of which are expressly incorporated herein by reference.

Some references, which may include patents, patent applications and various publications, are cited and discussed in the description of this invention. The citation and/or discussion of such references is provided merely to clarify the description of the present invention and is not an admission that any such reference is “prior art” to the invention described herein. All references cited and discussed in this specification are incorporated herein by reference in their entireties and to the same extent as if each reference was individually incorporated by reference.

### FIELD OF THE INVENTIONS

The present invention relates generally to light scattering and absorption, and in particular to probe apparatuses and component combinations thereof that are used to screen for possibly abnormal living tissue

### BACKGROUND

Optical probes are known that detect optical signals. Simple optical probes will transmit broadband or a laser light to a target with one optical fiber, and receive the light such as light that is elastically scattered from a specimen, fluorescent light, Raman scattered light, etc., with another optical fiber. The received backscattered light can be channeled to a receiver, such as a CCD array, and the spectrum of the signal is recorded therein.

While such probes work sufficiently for their intended purposes, new observations in terms of the type of measurements that are required for diagnostic purposes have required further enhancements and improvements.

### SUMMARY

The present inventions relates generally to light scattering and absorption, and in particular to probe apparatuses and component combinations thereof that are used to recognize possibly abnormal living tissue.

In one aspect, the embodiments described herein are directed toward an apparatus that emits broadband light obtained from a light source onto microvasculature of tissue, particularly in a mucosal tissue layer disposed within a human body, and receives interacted light that is obtained from interaction of the broadband light with the microvasculature for transmission to a receiver.

In another aspect, the embodiments described herein are directed toward a apparatus that emits broadband light obtained from a light source onto tissue disposed within a human body, particularly in a mucosal tissue layer disposed within a human body, and receives interacted light that is obtained from interaction of the broadband light with the microarchitecture tissue for transmission to a receiver.

In a particular aspect, a disposable, finger mounted optical probe is described.

In a further embodiment, an optical probe that contains a disposable tip with a retractable integral probe is disclosed.

Different further embodiments of both the disposable, finger mounted optical probe and the optical probe that contains the disposable tip with the retractable integral probe are described which include various combinations of optical fibers, polarizers and lenses that assist in the selection of a predetermined depth profile of interacted light for a variety of different wavelength ranges of light, and for different applications.

### BRIEF DESCRIPTION OF THE DRAWINGS

These and other aspects and features of the present invention will become apparent to those of ordinary skill in the art upon review of the following description of specific embodiments of the invention in conjunction with the accompanying figures, wherein:

FIGS. 1 and 2 illustrate a housing of a disposable, finger mounted optical probe according to one embodiment.

FIG. 3 illustrates a disposable tip and re-usable trunk usable in one embodiment of the disposable, finger mounted optical probe.

FIGS. 4(a)-(b) illustrate another embodiment of the disposable, finger mounted optical probe containing a pre-loaded optical assembly.

FIGS. 5a-5c are illustrations of the method of use of the disposable, finger mounted optical probe.

FIGS. 6A, B(1)-(2) and C show usage of an embodiment of an optical probe that contains a permanent housing and disposable tip with retractable integral optical fibers.

FIG. 7 illustrates a partial illustration of a particular embodiment of an optical probe that contains a permanent housing and a disposable tip assembly with a retractable integral optical fiber assembly.

FIG. 8 illustrates a partial illustration of another particular embodiment of an optical probe that contains a permanent housing and disposable tip assembly with a retractable integral optical fiber assembly.

FIG. 9 illustrates a particular embodiment of a disposable tip that includes a protective sheath that is used with the optical probe that contains a permanent housing and disposable tip assembly with a retractable integral optical fiber assembly.

FIG. 10 illustrates a partial illustration of a further particular embodiment of an optical probe that contains a permanent housing and disposable tip assembly with a retractable integral optical fiber assembly and an integral CCD module.

FIG. 11 illustrates a particular optical probe assembly configuration used for EIBS.

FIG. 12 illustrates another particular optical probe assembly configuration used for EIBS.

FIG. 13 illustrates a further particular optical probe assembly configuration used for EIBS.

FIG. 14 illustrates in cross section an embodiment of optical fibers and polarizer usable in the optical probe assembly configurations illustrated in any of FIGS. 11, 12, and 13.

FIG. 15 illustrates in cross section a further embodiment of optical fibers and polarizer usable in the optical probe assembly configurations illustrated in any of FIGS. 11, 12, and 13.

FIG. 16 illustrates a particular optical probe assembly configuration used for LEBS.

FIG. 17 illustrates another particular optical probe assembly configuration used for LEBS.

FIG. 18 illustrates a further particular optical probe assembly configuration used for LEBS.

FIG. 19 illustrates a further particular optical probe assembly configuration used for LEBS.

FIG. 20 illustrates a further particular optical probe assembly configuration used for LEBS.

FIGS. 21(a) and (b) illustrate in cross section an embodiment of optical fibers usable in the optical probe assembly configurations illustrated in any of FIGS. 16-20.

FIG. 22 illustrates in cross section a further embodiment of optical fibers usable in the optical probe assembly configurations illustrated in any of FIGS. 16-20.

### DETAILED DESCRIPTION OF THE PREFERRED EMBODIMENTS

The present inventions are more particularly described in the following examples that are intended as illustrative only since numerous modifications and variations therein will be apparent to those skilled in the art. Various embodiments are now described in detail. Referring to the drawings, like numbers indicate like components throughout the views. As used in the description herein, the meaning of “a”, “an”, and “the” includes plural reference unless the context clearly dictates otherwise. Also, as used in the description herein and throughout the claims that follow, the meaning of “in” includes “in” and “on” unless the context clearly dictates otherwise. Moreover, titles or subtitles may be used in the specification for the convenience of a reader, which shall have no influence on the scope of the present invention. Additionally, some terms used in this specification are more specifically defined below.

The terms used in this specification generally have their ordinary meanings in the art, within the context of the invention, and in the specific context where each term is used. Certain terms that are used to describe the invention are discussed below, or elsewhere in the specification, to provide additional guidance to the practitioner regarding the description of the invention, For convenience, certain terms may be highlighted, for example using italics and/or quotation marks. The use of highlighting has no influence on the scope and meaning of a term; the scope and meaning of a term is the same, in the same context, whether or not it is highlighted. It will be appreciated that same thing can be said in more than one way. Consequently, alternative language and synonyms may be used for any one or more of the terms discussed herein, not is any special significance to be placed upon whether or not a term is elaborated or discussed herein. Synonyms for certain terms are provided. A recital of one or more synonyms does not exclude the use of other synonyms. The use of examples anywhere in this specification including examples of any terms discussed herein is illustrative only, and in no way limits the scope and meaning of the invention or of any exemplified term. Likewise, the invention is not limited to various embodiments given in this specification.

Unless otherwise defined, all technical and scientific terms used herein have the same meaning as commonly understood by one of ordinary skill in the art to which this invention pertains. In the case of conflict, the present document, including definitions will control.

As used herein, “around”, “about” or “approximately” shall generally mean within 20 percent, preferably within 10 percent, and more preferably within 5 percent of a given value or range. Numerical quantities given herein are approximate, meaning that the term “around”, “about” or “approximately” can be inferred if not expressly stated.

The present invention, in one aspect, relates to a probe apparatus that is used for optically screening a target for tumors or lesions. Various targets and corresponding optical probe types are disclosed, as well as various different probe housing designs are disclosed, and combination of them can be used interchangeably. Certain of the optical probe designs are for use in detecting what is referred to as “Early Increase in microvascular Blood Supply” (EIBS) that exists in tissues that are close to, but are not themselves, the lesion or tumor. Other of the LEBS (Low-coherence Enhanced Backscattering) optical probe designs are for use in detecting backscattered light that results from the interaction of low-coherent light with abnormal scattering structures in the microarchitecture of the tissue that exist in tissues that are close to, but are not themselves, the lesion or tumor. Both of these optical probe types, which have been described in applications previously filed and which are, as a result, known. As will be described herein, whether detection is made using the techniques associated with EIBS or LEBS probes and microarchitecture of the tissue, the probes as described herein, while normally made for usage with one of these techniques, will have aspects that are common between them.

One difference between a probe that detects EIBS and an LEBS probe that detects tissue microarchitecture is that with an probe that detects EIBS, data from a plurality of depths can be obtained in one measurement by looking at co-pol and cross-pol and co-pol minus cross-pol received signals, whereas for an LEBS probe, only one depth is obtained for a specific configuration.

A particular application described herein is for detection of such lesions in colonic mucosa in early colorectal cancer (“CRC”), but other applications such as pancreatic cancer screening are described as well.

The target is a sample related to a living subject, particularly a human being. The sample is a part of the living subject, such that the sample is a biological sample, wherein the biological sample may have tissue developing a cancerous disease.

The neoplastic disease is a process that leads to a tumor or lesion, wherein the tumor or lesion is an abnormal living tissue (either premalignant or cancerous), which for the probes described herein is typically a colon cancer, an adenomatous polyp of the colon, or other cancers.

The measuring step is performed in vivo using the probes described herein and may further comprise the step of acquiring an image of the target. The image, obtained at the time of detection, can be used to later analyze the extent of the tumor, as well as its location.

In the various embodiments, the probe projects a beam of light to a target that has tissues and/or blood circulation associated therewith, depending upon the target type. Light scattered from the target is then measured, and target information is obtained from the measured scattered light. The obtained target information can be information for the targets as described in the patent applications incorporated by reference above, as well as the data related to blood vessel size and oxygenated hemoglobin as described in U.S. patent application Ser. No. 12/350,955 filed Jan. 8, 2009 entitled “Method Of Screening For Cancer Using Parameters Obtained By The Detection Of Early Increase In Microvascular Blood Content” filed on this same day, bearing Attorney Docket Number 042652-0376943.

The beam of light projected is obtained from a light source that may comprise an incoherent light source (such as a xenon lamp, light emitting diode, etc).

In all of the embodiments described herein, there is at least one first type fiber comprises an illumination fiber, wherein the illumination fiber is optically coupled to the light source.

There is also at least one second type fiber formed with one or more collection fibers, wherein the one or more collection fibers are optically coupled to a detector, such as an imaging spectrograph and a CCD at the distal end portion, which imaging spectrograph is used to obtain an image of the target and obtain detected data therefrom.

The following further details of the preferred embodiments that will further describe the invention. Without intent to limit the scope of the invention, exemplary instruments, apparatus, methods and their related results according to the embodiments of the present invention are given below. Note that titles or subtitles may be used in the examples for convenience of a reader, which in no way should limit the scope of the invention. Moreover, certain theories are proposed and disclosed herein; however, in no way they, whether they are right or wrong, should limit the scope of the invention so long as the invention is practiced according to the invention without regard for any particular theory or scheme of action.

The optical probes described herein can be used in-vivo to take optical measurements of tissue, such as just inside the rectum to assess a patient's risk of colon cancer. If rectal, the rectally inserted probe for analysis of rectal mucosa provides a means of assessing a patient's risk of developing colon cancer without the need for colonoscopy or colon purging.

In order to facilitate the acquisition of such a measurement, the probes described herein are necessarily introduced into a patient's colorectal vault via an insertion device such as a colonoscope, an upper GI therapeutic scope (a device which is generally known), a disposable, finger mounted device, or an optical probe that contains a permanent housing and disposable tip with retractable integral optical fibers, the latter of which are further described herein.

For clinical evaluation of a colon, the probe is inserted into the rectum to establish contact with the colorectal mucosal wall, perform optical measurements as needed, and is then removed. The probes described further herein provide an insertion device for guiding the probe on a pathway through the rectum to reach the colo-rectal mucosal wall, while shielding the probe tip from possible blockage caused by loose stool that the probe may encounter. While contacting the colorectal mucosal wall, the insertion device then allows the optical portion of probe to extend some distance out of the tip of the insertion device and perform optical measurements as needed.

The optical probes with insertion devices as described further herein contain components that are partially or entirely disposable, since for health reasons certain components are not readily used in multiple different patients.

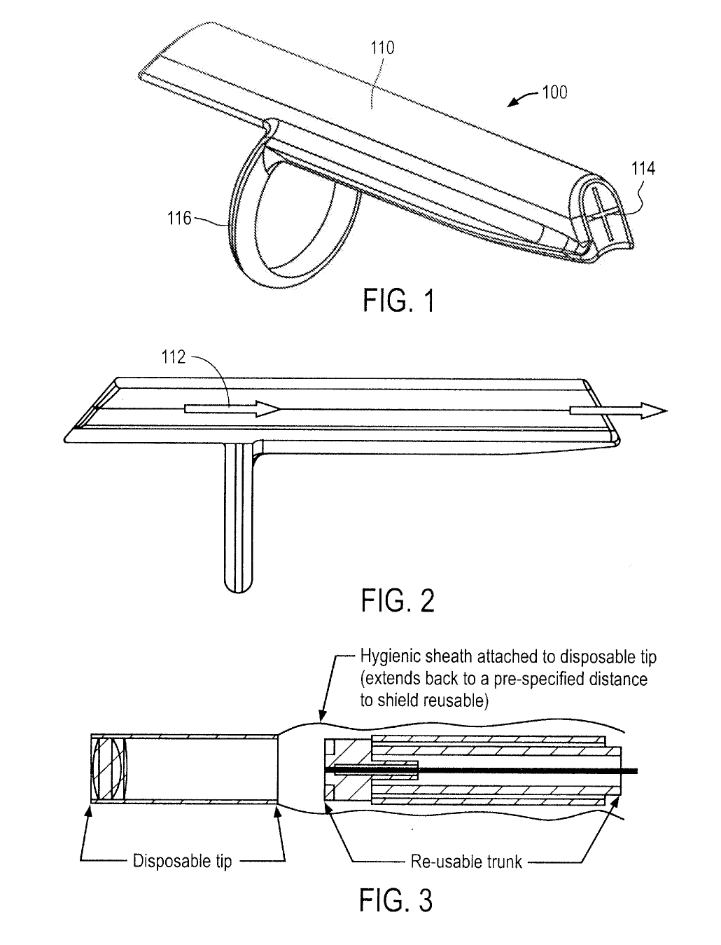

FIGS. 1-3 illustrate a housing 110 of a disposable, finger mounted optical probe 100 according to one embodiment, which is a semi-flexible component that includes a finger loop 116 worn over the physicians finger. As shown in FIG. 3, incorporated within the housing 110 is a complete optical probe 120, including a re-usable trunk 140 and disposable tip 130, described further herein, which are connected together by some type of engagement mechanism, such as threads on both the tip assembly 130 and the trunk assembly 140. This finger mounted optical probe 100 is inserted into the patient's rectum mounted on the finger of the physician, allowing for passage of the optical probe 120 to the mucosal wall for measurement acquisition while shielding from potential loose stool both the optical probe, and particularly the optical components of the optical probe 120 that are disposed within the disposable tip 130.

The housing 110 of the disposable, finger mounted optical probe 100 is sufficiently lubricious to provide for easy passage of optical fibers through internal lumen 112, and on its outer surface for non-lubricated device insertion into a patient's rectum. The housing may be made of liquid injection molded silicone rubber or similar material. Further, a parylene-N coating may be added to some or all surfaces of the housing 110 to increase overall lubricity for ease of feeding of probe through inner lumen, and insertion into the patient.

The outer front surface of the housing 110 preferably includes a perforated membrane 114 that shields the probe tips from loose stool that may be encountered within the patient, through which the probe tip can pass through just prior to acquisition of optical measurement on the mucosal wall, as described herein, though such a perforated membrane 114 is not necessarily needed.

Further, the disposable, finger mounted optical probe 100 will preferably either have: 1) a pre-formed geometry/curvature such that it can be guided to the proper location in the colo-rectal mucosal anatomy, 2) sufficient flexibility such that the physician can bend and/or manipulate it to the same area for optical measurement, or 3) some combination of both aforementioned attributes. If preformed, the probe 100 preferably has flexibility such that it could be inserted in a straight fashion, and shape memory such that it would retake its original shape once fully inserted into patient's colorectal vault.

The probe 100 as illustrated in FIG. 1-3 allows for pass through of a fully assembled optical probe. This embodiment require the disposable tip 130 to be attached to the reusable trunk 140 prior to insertion. The disposable tip 130 is clean or sterile when initially used prior to insertion, and also includes attached thereto a hygienic sheath 150 that acts as a hygienic shield to cover the reusable trunk 140, which need not be sterile or sterilized when used. The hygienic sheath 150 may be made of a sterile thin polyethylene film or similar material.

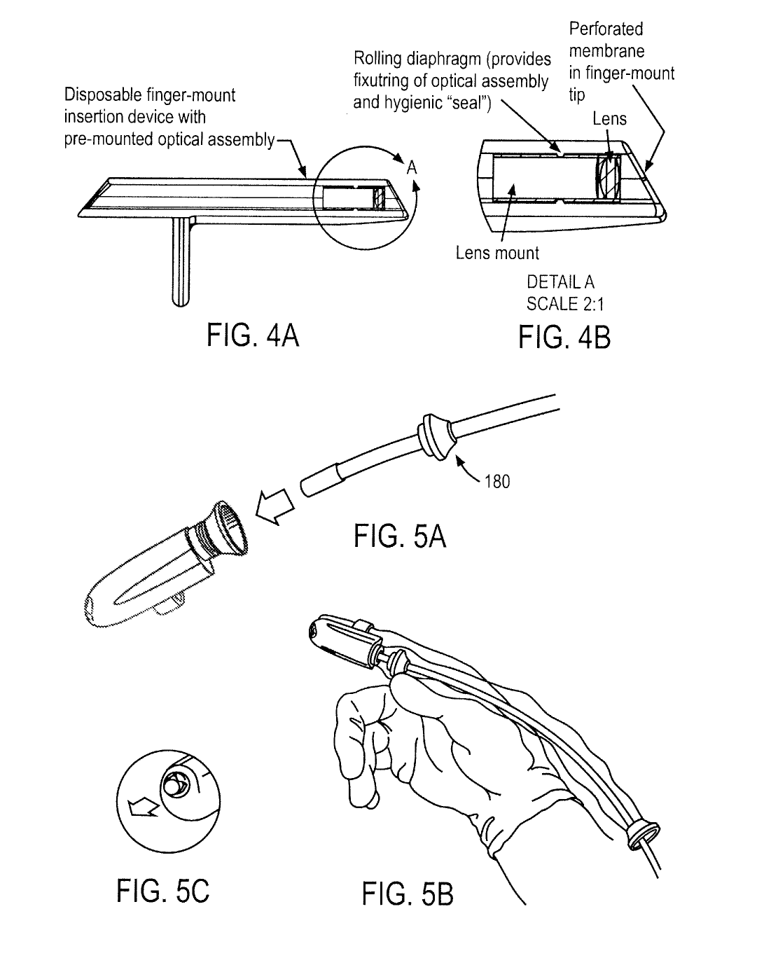

FIGS. 4(a)-(b) illustrate another embodiment of the disposable, finger mounted optical probe 100A containing a pre-loaded optical assembly. In this embodiment, the housing 110 and the lumen 112 therein provides for pre-loading of an optical assembly 160, such that the re-usable trunk (as described with reference to FIG. 3) will connect to the optical assembly 160 (essentially the same as the disposable tip 130) within the lumen 112, and the entire assembly, once connected, can then continue to be positioned by moving through the lumen 112, and eventually out through any perforated membrane 114. As shown in FIG. 4(b), the optical assembly, in one embodiment, may include a lens mount 162, a rolling diaphragm 164 that provides fixturing of the optical assembly and a hygienic seal This hygienic seal can be simply a narrowing of the lumen such that the lens mount 162 fits tightly around the optical assembly to prevent fluid from flowing backward but is not so tight as to prevent the optical assembly from sliding forward and back, and a lens 166, though other components, such as polarizers and spacers, can also be used within optical assembly 160.

In the embodiment of FIG. 4, the hygienic sheath is preferably attached to the disposable housing 110 at the entry end 118 of the housing, though the sheath is not shown in the Figure, though it could also be attached within the lumen 112 and be part of the optical assembly 160 to address the possibility of cross-contamination. This sheath would extend back to cover all non-disposable surfaces of the probe assembly which may be manipulated by the physician. The finger-mounted insertion device 100A is preferably entirely disposable, and intended for single-use. An advancement assist ring 116 may be permanently attached to the optical probe to facilitate single handed probe insertion.

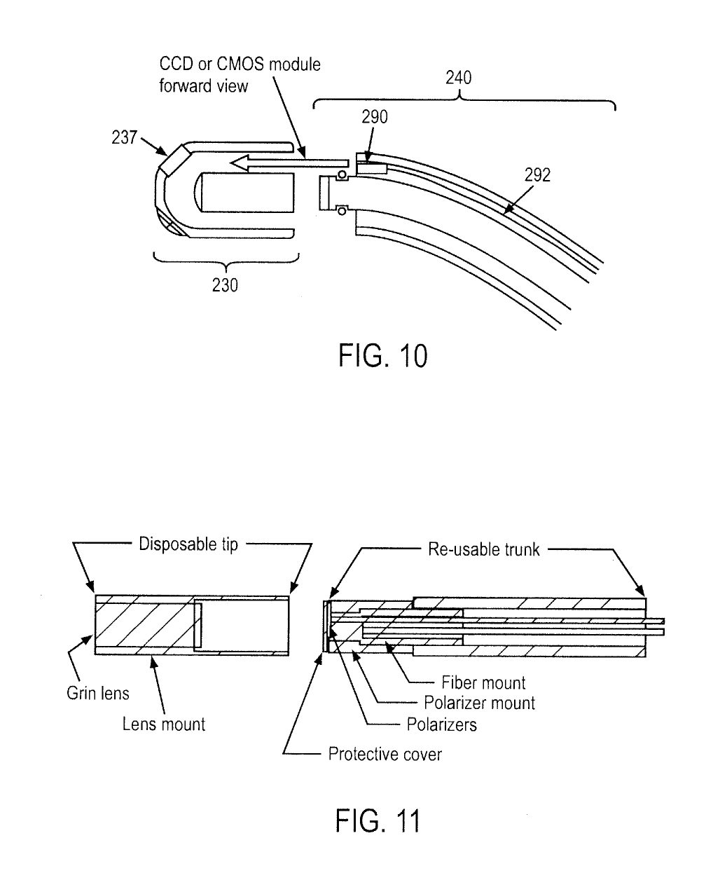

Measurement acquisition may be initiated by a foot pedal connected to an instrumentation unit, a button built into the reusable portion of the probe assembly, or some other mechanism. If blind measurement acquisition and/or insertion is not deemed acceptable, a forward viewing CCD or CMOS camera module may be designed into the device, with camera residing in the reusable probe trunk, and window built into the disposable insertion device, as shown in FIG. 10.

FIGS. 5a-5c are illustrations of the method of use of the disposable, finger mounted optical probe 100. In use, the probe assembly 120, formed of the re-usable trunk 140 and the disposable tip 130, is inserted into the housing 110 as shown, and an advancement assist ring 180, permanently attached to the re-usable trunk 140, will attach to the end 118 of the housing 110. As shown in FIGS. 5A and 5B, the sheath 150 is pulled back so that it extends sufficiently below the sterile gloved hand of the physician to provide a sterile environment for the patient. As shown in FIG. 5C, the disposable tip 130 of the probe assembly 120 is pushed through the perforated membrane 114 at the time the measurement is taken.

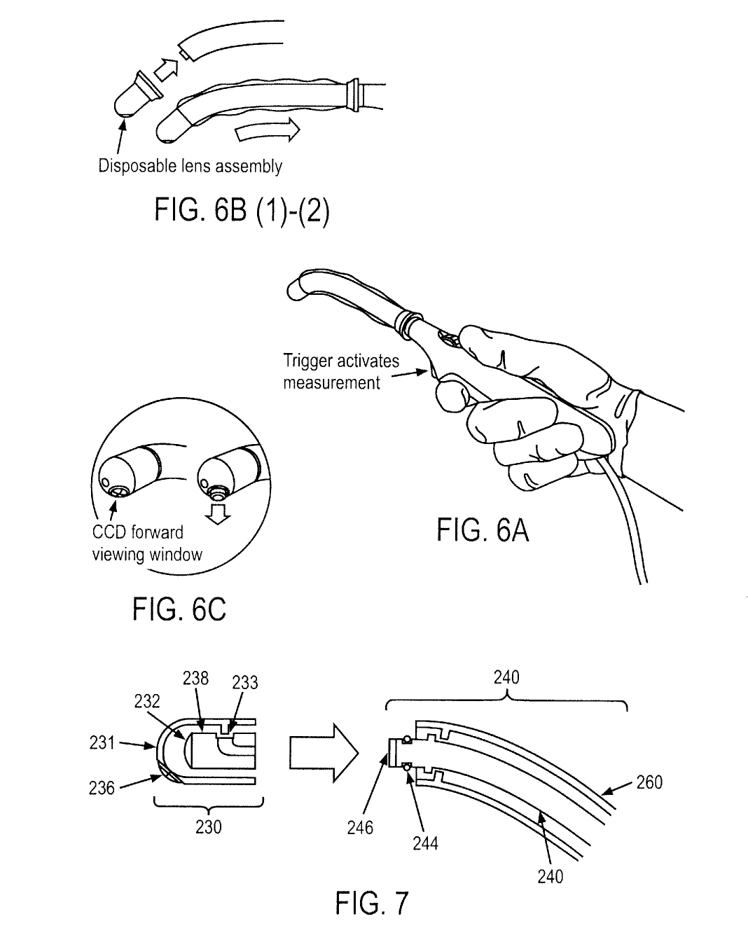

FIGS. 6A(1)-(2), B and C show usage of an embodiment of an optical probe 200 that contains a permanent housing 210 and a disposable tip assembly 220 with retractable integral optical fiber assembly 220 (essentially the same as the optical assembly 120 that is formed of the disposable tip 130 and the re-usable trunk 140 as described in the FIG. 3 embodiment above), as well as an overall view of this embodiment. In all of the embodiments there exist the permanent housing 210, which preferably includes thereon a trigger activation button 212, a grip 214 for holding in the physician hand, and a roller wheel 216 or similar element integrated into the housing 210 to facilitate single-handed probe advancement, as shown in FIG. 6A. FIGS. 6B1 and 6B2 show at a high level both the connection of the disposable tip assembly 230 to the re-usable trunk assembly 240, as well as the unwrapping of the protective sheath 250 over the exterior of the housing 210. It is noted that in FIG. 6A the sheath 250 is only shown unrolled on the insertion portion 260, but preferably the sheath 250 will extend below the entire housing 210. FIG. 6C provides close up views of the disposable tip assembly 230, and shows both a CCD forward viewing window 270 for a CCD array disposed therebehind (not shown here, though components illustrated in FIG. 10 can work herein), as well as the perforated membrane 280 through which the disposable tip 220 assembly will be moved when the measurement is taken. In use, the insertion portion 260 is inserted into the patient's rectum, with the grip 214 of the housing 210 held by the physician, allowing for internal optical assembly to be positioned on the mucosal wall while shielded from potential loose stool. This allows for advancement of the internal optical probe assembly, including the lens as described hereinafter, out of the protective cap associated with the disposable tip assembly 220, and onto the patient's colo-mucosal wall for measurement acquisition.

In a preferred implementation, the housing 210 a two-piece, rigid injection molded handle comprised of ABS (Acrylonitrile butadiene styrene) or similar material. Further, an overmolded soft-touch material such as Pebax or Hytrel may comprise the insertion portion 260. The disposable tip assembly 230 in this configuration may be comprised of a similar soft-touch material overmolded soft-touch material such as Pebax or Hytrel. The hygienic sheath 250 attached to the lens mount 238 within disposable tip assembly 230 may be made of a thin polyethylene film or similar material.

It is noted that it may be that a sheath 250 isn't used, and the insertion portion 260 is sterilized after each use. In such a use, the insertion portion 260 is preferably lubricious enough on its outer surfaces for non-lubricated device insertion into a patient's rectum.

Further, this probe 200 also preferably has 1) a pre-formed geometry/curvature such that it locates the internal optical assembly, and particularly the optical tip, onto proper location in the colo-rectal mucosal anatomy, and 2) sufficient flexibility such that the physician could bend and/or manipulate the device to the same area for optical measurement. The probe 200 is sufficiently flexible such that it can be inserted in a straight fashion, and has shape memory such that it retakes its original shape once fully inserted into patient's colorectal vault.

FIG. 7 illustrates a partial illustration of a particular embodiment of an optical probe 200A, with only the optical components shown, not the sheath 250 and lower part of the housing 210. The shown semi-flexible insertion portion 260 contains therein the retractable integral optical fiber assembly 220, formed of the disposable tip assembly 230 and the trunk assembly 240. As shown the trunk assembly 240 will contain an outer sheath 248, which preferably includes at the distal end a protrusion ring 242, which abuts a similar protrusion ring 262 associated with the insertion portion of the housing 210. Also associated with the re-usable trunk assembly 240 is a springing engaging mechanism 244 for the optical components of the disposable tip assembly 230 to connect in an aligned manner, as well as, in certain configurations, other optical components 246, such as a polarizer or protective cover. Other engagement mechanism, such as threads on both the tip assembly 230 and the trunk assembly 240 can be used.

The disposable tip assembly 230 contains a protective cap 231 that has an alignment element 233 and perforated membrane 236, described further herein, that maintains the lens mount 238 in place prior to connection to the optical fiber trunk assembly 240. As shown in FIG. 9, the disposable tip assembly also preferably has attached thereto the sheath 250

The lens mount 238 will contain a lens 232, such as a GRIN lens, a ball lens, an achromatic doublet lens, etc can be used, disposed therein or as part of a one-piece assembly, as well as an alignment member 234 that engages with the alignment element 233. The alignment member 234 in one embodiment is a channel into which a protrusion that is the alignment element 233 fits. Once the disposable tip assembly 230, and specifically the lens mount 238, is connected to the trunk assembly 240, and the engaging mechanism 244, the entire optical assembly 220 is moved through the rectum to the measurement point. At that time, the optical fiber assembly 220 can be slightly rotated and moved forward, so that the lens mount 238, via the alignment member 234, is guided by the alignment element 233, so that the lens 232 can protrude through the perforated membrane 236.

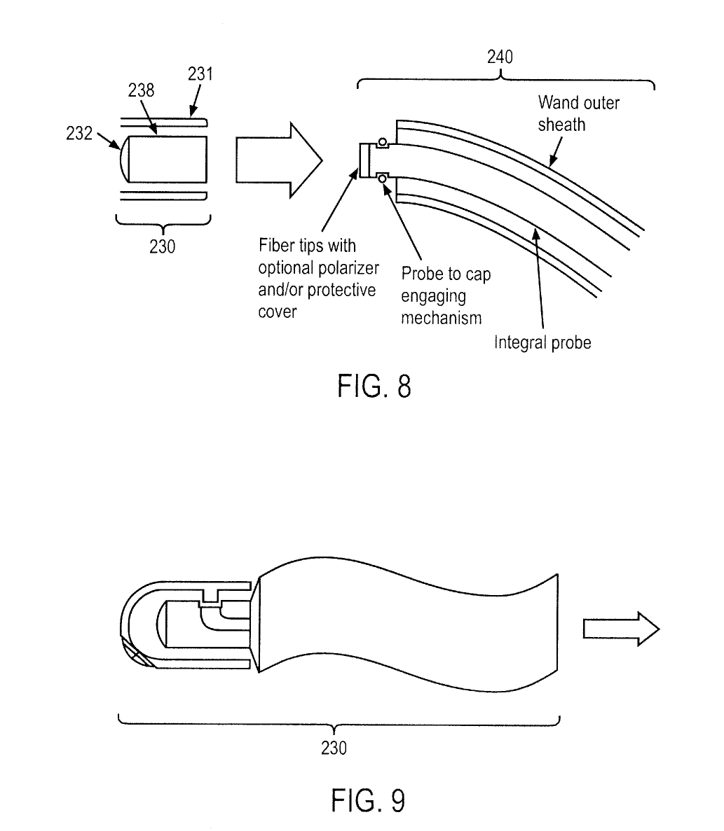

FIG. 8 illustrates a partial illustration of a particular embodiment of an optical probe 200B, with only the optical components shown, not the sheath 250 and lower part of the housing 210. In this embodiment, as shown the disposable tip assembly 230 does not contain a front face to the protective cover 231 or a perforated member, and as such the lens 232, mounted in the lens mount 238, is exposed. Otherwise, the elements shown in FIG. 8 are the same as those described previously with respect to FIG. 7. Since the lens 232 is pre-exposed, the probe 200B does not required advancement of retractable integral optical fiber assembly 220 to break through any protective cap membrane. Thus, once inserted and put into contact with the patient's colo-mucosal wall, the probe 200B is immediately ready for measurement acquisition.

If blind insertion is not deemed acceptable, a forward viewing CCD camera may be designed into the device, with camera residing in the tip of reusable portion of the wand, and window built into the disposable wand tip, as shown in FIG. 10. As shown, the disposable tip assembly 230 is modified by including the glass viewing cover 237 as part of the protective cap 231, and the probe 200 further includes a CCD or CMOS module, as will as an image return wiring 292 as needed. Depending on the configuration, the CCD or CMOS module may include battery power, may be powered via wires for the power, and/or the power and/or image signals may be transmitted wirelessly using various conventional data and short range power transmission schemes.

Different penetration depths are implemented with these probes in a variety of ways. Different fibers and/or disposable tips can be used (in some instances with different probes, in other instances all within the same probe) in order to achieve the desired results. For probes that detect EIBS in particular, the choice of the spacing between the fiber termination and lens (e.g. nominally 1 focal length but could be more or less) and selection of the lens type and focal length adjustment depth can be used to achieve different penetration depth. For LEBS probes that detect tissue microarchitecture, the selection of the lens and the distance from the termination of the fibers to the lens or the length of the glass spacer determine the special coherence length of light, which will vary the penetration depth.

In use, depending upon the target and the application, each probe may take multiple measurements, and the detected data from each measurement stored for subsequent usage. Typically a number of different measurement locations, such as 3-6, but not typically greater than 10 will be made. Depending on the probe or the manner in which the probe is used, various different penetration depths may then be sensed at each measurement location.

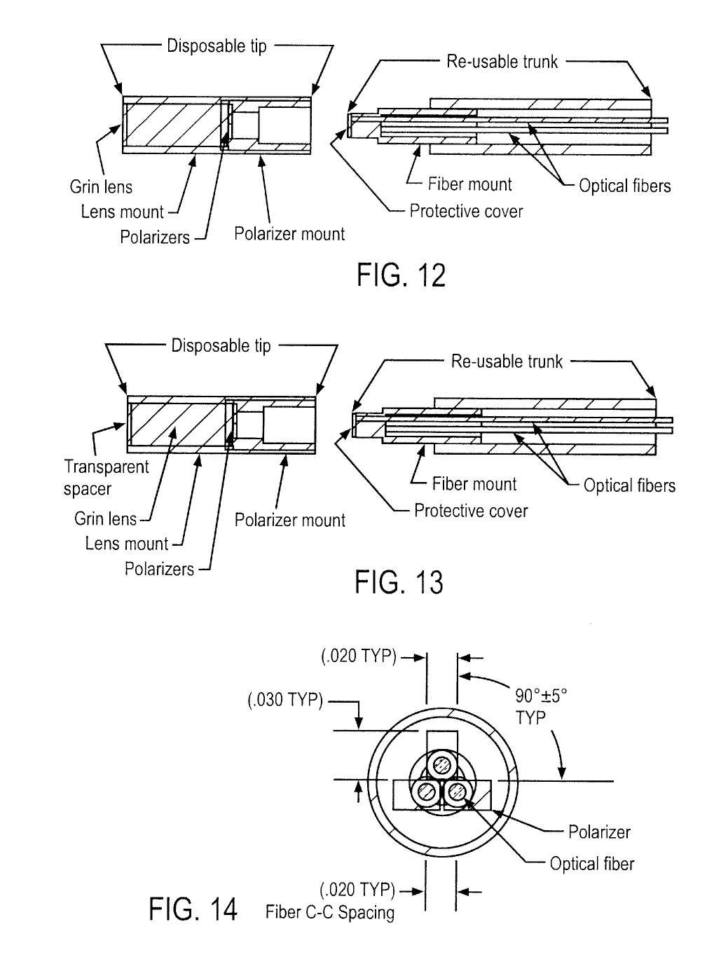

FIG. 11 illustrates a particular optical probe assembly configuration used for EIBS. FIG. 12 illustrates another particular optical probe assembly configuration used for EIBS. It is noted that the lens mount and polarizer mount may be combined to form a single component. FIG. 13 illustrates a further particular optical probe assembly configuration used for EIBS. It is noted that the lens mount and polarizer mount may be combined to form a single component. In each of FIGS. 11, 12 and 13, the components are identified, and they together show that various combinations of components can be used: certain embodiments may or may not have polarizers, spacers and different numbers of optical fibers can also be used. In this regard, reference is made to the previously filed U.S. patent application Ser. No. 11/604,659 filed Nov. 27, 2006 and entitled “Apparatus For Recognizing Abnormal Tissue Using The Detection Of Early Increase In Microvascular Blood Content.”

FIG. 14 illustrates in cross section an embodiment of optical fibers and polarizer usable in the optical probe assembly configurations illustrated in any of FIGS. 11, 12, and 13.

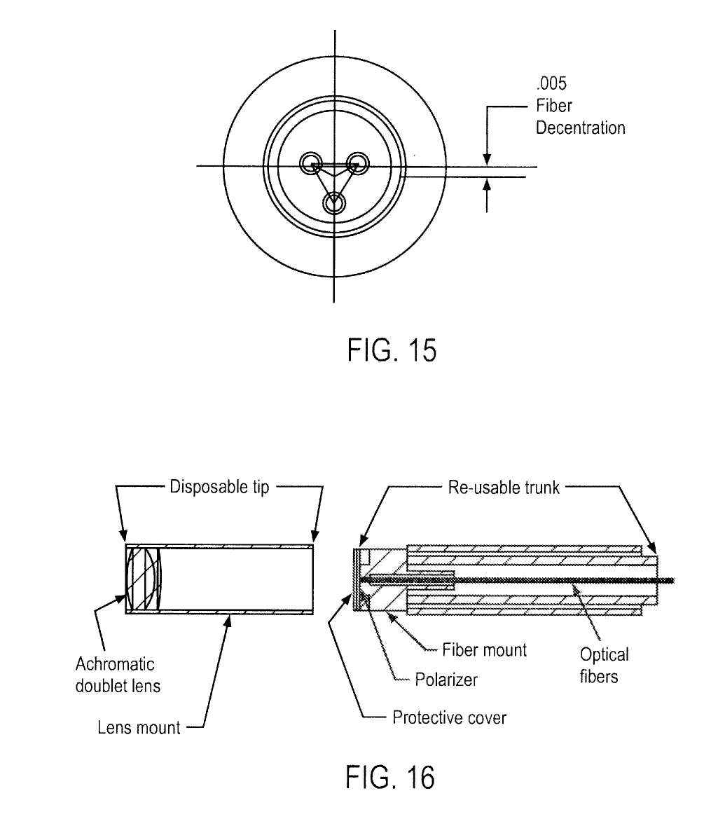

FIG. 15 illustrates in cross section a further embodiment of optical fibers and polarizer usable in the optical probe assembly configurations illustrated in any of FIGS. 11, 12, and 13, and shows a decentering or making the fibers slightly asymmetric with respect to the probe center to minimize reflections. This could be used on any probe designs that detect EIBS described herein.

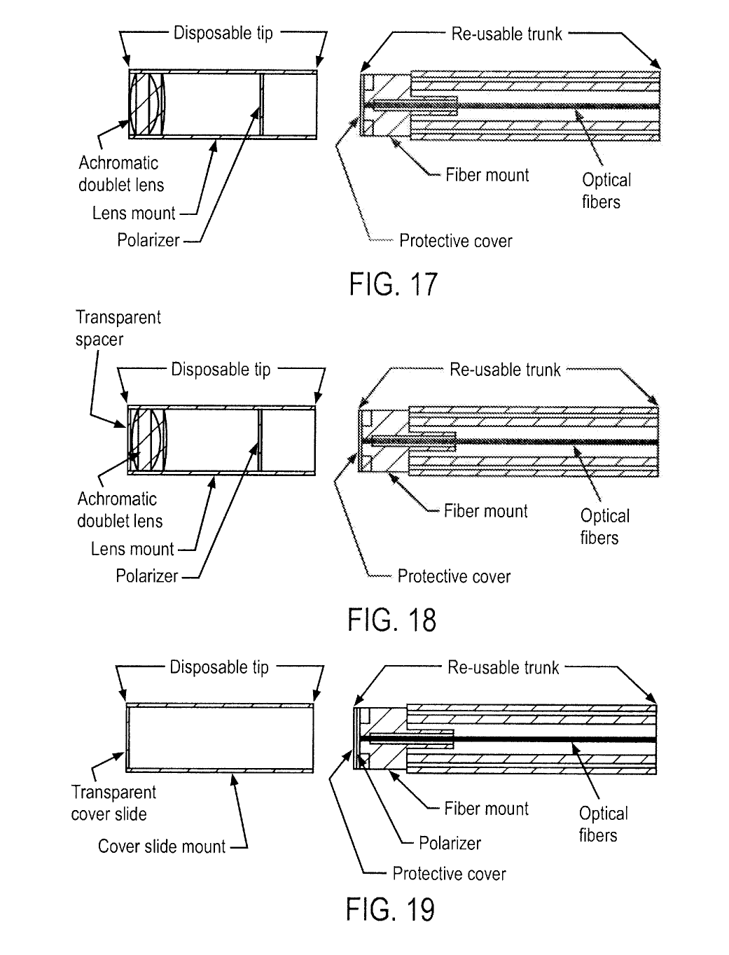

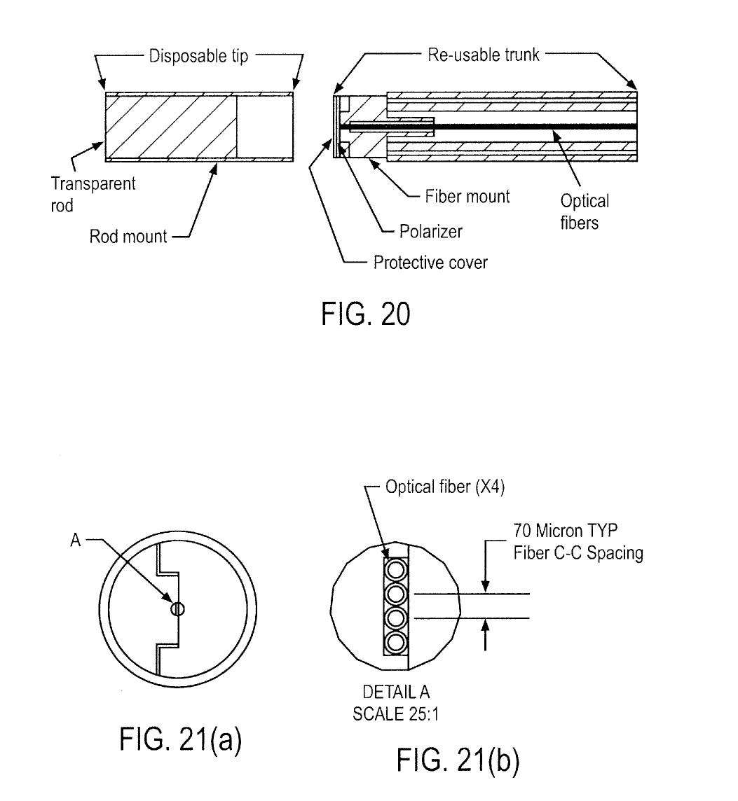

FIG. 16 illustrates a particular optical probe assembly configuration used for LEBS. FIG. 17 illustrates another particular optical probe assembly configuration used for LEBS. FIG. 18 illustrates a further particular optical probe assembly configuration used for LEBS. FIG. 19 illustrates a further particular optical probe assembly configuration used for LEBS. FIG. 20 illustrates a further particular optical probe assembly configuration used for LEBS. In both of the FIG. 19 and FIG. 20 probe designs, no lens is used but the solid glass spacer (FIG. 20) or air gap with coverglass (FIG. 19) between the fiber terminations and the tissue selects a specific (and predetermined) spatial coherence length that corresponds to a desired depth. This lensless concept that uses a fix-distance spacer (air or glass) can be used to establish a spatial coherence length. In the other embodiments, the components are identified, and they together show that various combinations of components can be used: certain embodiments may or may not have polarizers, spacers and different numbers of optical fibers can also be used.

FIGS. 21(a) and (b) illustrate in cross section an embodiment of optical fibers usable in the optical probe assembly configurations illustrated in any of FIGS. 16-20.

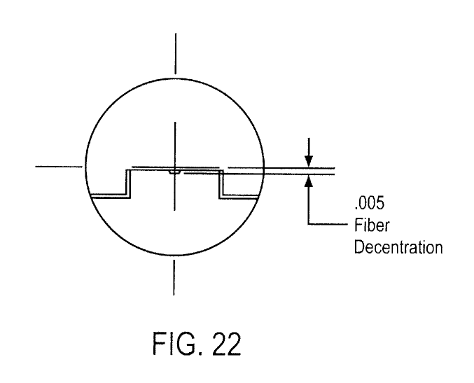

FIG. 22 illustrates in cross section a further embodiment of optical fibers usable in the optical probe assembly configurations illustrated in any of FIGS. 16-20. FIG. 22 shows a decentering or making the fibers slightly asymmetric with respect to the probe center to minimize reflections. This could be used on any LEBS probe designs described herein. This gives a potential advantage in that internal reflections off surfaces (e.g. the lens/tissue interface, air/lens interface, etc) will be reflected elsewhere away from the fibers.

The foregoing description of the exemplary embodiments of the invention has been presented only for the purposes of illustration and description and is not intended to be exhaustive or to limit the invention to the precise forms disclosed. Many modifications and variations are possible in light of the above teachings.

What is claimed is:

1. An apparatus that emits broadband light obtained from a light source onto one of microvasculature of tissue of a human body and tissue microarchitecture within a cavity of the human body, and that receives interacted light that is obtained from interaction of the broadband light with the one of the microvasculature and tissue microarchitecture for transmission to a receiver, the apparatus comprising:
a probe having an end adapted for insertion into the human body and which illuminates the tissue with the broadband light and receives interacted light that interacts with blood in the one of the microvasculature and tissue microarchitecture that is within the tissue, the probe including:
an integrated optical fiber assembly that includes:
a delivery optical fiber having a delivery numerical aperture for transmitting the broadband light obtained from the light source, the delivery optical fiber having a light delivery end adapted for emission of the broadband light and a light delivery source connection end adapted for connection to the light source; and
at least one collection optical fiber having a collection numerical aperture, the collection optical fiber having a light collection end that receives the interacted light and a receiver connection end adapted for connection to the receiver, wherein the light collection end is substantially aligned with and at a predetermined distance from the light delivery end of the delivery optical fiber; and
a tip assembly that releasably connects to the integrated optical fiber assembly, the tip assembly including:
a housing;
an optical component disposed at least partially within the housing and having a tip surface that is arranged in a predetermined locational relationship, when the integrated optical fiber assembly is connected to the tip assembly, with the light collection end of the collection optical fiber and the light delivery end of the delivery optical fiber, wherein the optical component and the predetermined locational relationship are selected to provide a predetermined penetration depth of the emitted broadband light below a collection spot on a surface of the tissue when the tip surface rests on the surface of the tissue; and
a hygienic sheath having a tip end and another end with a sheath body therebetween, such that the tip end is disposed within the housing and the sheath body covers portions of the delivery optical fiber and the at least one collection optical fiber that are inserted within the cavity of the body, thereby maintaining a clean environment; and
wherein the collection optical fiber and the optical component are adapted to collect the interacted light at the collection spot, and wherein the interacted light collected results from interactions with the one of the microvasculature and the tissue microarchitecture disposed substantially at the predetermined penetration depth below the collection spot.

2. The apparatus according to claim 1 wherein the housing of the tip assembly further includes a perforatable membrane, such that the tip surface of the optical component is movable through the perforatable membrane and onto the surface of the tissue when the tip assembly is in a vicinity of the surface of the tissue.

3. The apparatus according to claim 2 further including at least one alignment member that guides movement of the optical component through the perforatable membrane.

4. The apparatus according to claim 2 wherein at least some of the delivery optical fiber and the at least one collection optical fiber are moveable through the housing.

5. The apparatus according to claim 1 wherein the housing includes a finger loop such that the tip assembly is insertable into the cavity using a finger in the finger loop.

6. The apparatus according to claim 1, wherein the optical component is at least a lens, and wherein a center of the delivery optical fiber and the at least one collection optical fiber are asymmetric relative to a center of the lens to minimize backwards reflected light.

7. The apparatus according to claim 1 wherein the tip assembly further includes one of a CCD array and CMOS camera mounted within the housing.

8. The apparatus according to claim 1 wherein the optical component is one of a lens and spacer, and a surface of the one of the lens and the spacer is the tip surface.

9. The apparatus according to claim 8 wherein the optical component is a lens, and wherein the optical component further includes a polarizer.

10. The apparatus according to claim 8 wherein there is included a least two collection optical fibers.

11. The apparatus according to claim 1 wherein the predetermined penetration depth is less than less than 300 um.

12. The apparatus according to claim 1 wherein the predetermined penetration depth is less than less than 100 um.

13. An apparatus that connects to an integrated optical fiber assembly that includes a delivery optical fiber having a light delivery end for delivering emitted broadband light and at least one collection optical fiber having a light collection end to collect interacted light, wherein the emitted broadband light illuminates one of microvasculature of tissue of a human body and tissue microarchitecture within a cavity of the human body, the apparatus comprising:
a tip assembly that releasably connects to the integrated optical fiber assembly, the tip assembly including:
a housing;
an optical component disposed at least partially within the housing and having a tip surface that is arranged in a predetermined locational relationship, when the integrated optical fiber assembly is connected to the tip assembly, with the light collection end of the collection optical fiber and the light delivery end of the delivery optical fiber, wherein the optical component and the predetermined locational relationship are selected to provide a predetermined penetration depth of the emitted broadband light below a collection spot on a surface of the tissue when the tip surface rests on the surface of the tissue; and
a perforatable membrane formed on the housing, such that the tip surface of the optical component is movable through the perforatable membrane and onto the surface of the tissue when the tip assembly is in a vicinity of the surface of the tissue.

14. The apparatus according to claim 13 further including:
a hygienic sheath having a tip end and another end with a sheath body therebetween, such that the tip end is disposed within the housing and the sheath body covers portions of the delivery optical fiber and the at least one collection optical fiber that are inserted within the cavity of the body when the integrated optical fiber assembly is connected to the tip assembly, thereby maintaining a clean environment.

15. The apparatus according to claim 13 further including at least one alignment member that guides movement of the optical component through the perforatable membrane.

16. The apparatus according to claim 13 wherein the housing includes a finger loop such that the tip assembly is insertable into the cavity using a finger in the finger loop.

17. The apparatus according to claim 13 wherein the tip assembly further includes one of a CCD array and CMOS camera mounted within the housing.

18. The apparatus according to claim 13 wherein the optical component is one of a lens and spacer, and a surface of the one of the lens and the spacer is the tip surface.

19. The apparatus according to claim 18 wherein the optical component is a lens, and wherein the optical component further includes a polarizer.

20. The apparatus according to claim 13 wherein the predetermined penetration depth is less than less than 300 um.

21. The apparatus according to claim 13 wherein the predetermined penetration depth is less than less than 100 um.

22. A obtaining an optical measurement at a tissue surface within a cavity of a human body comprising the steps of:
providing a tip assembly having an optical component with a tip surface disposed within a housing that has a perforatable membrane thereon, the optical component of the tip assembly being optically coupled to a light collection end of a collection optical fiber and a light delivery end of a delivery optical fiber;
inserting the tip assembly into the cavity within a vicinity of the tissue surface; and
moving the optical component through the perforatable membrane such that the tip surface perforates the perforatable membrane and rests on a collection spot of the tissue surface;
illuminating the tissue with broadband light projected through the optical component;
obtaining the optical measurement at a predetermined penetration depth below the collection spot on the surface of the tissue when the tip surface rests on the collection spot of the tissue surface.

23. The method according to claim 22 further including the steps of:
removing the tip assembly from the light collection end of the collection optical fiber and the light delivery end of a delivery optical fiber;
providing another tip assembly having another optical component with another tip surface disposed within another housing that has another perforatable membrane thereon;
optically coupling the another optical component of the another tip assembly to the light collection end of the collection optical fiber and the light delivery end of the delivery optical fiber; and
repeating the steps of inserting, moving, illuminating and obtaining using the another tip assembly on another cavity of another human body.

24. The method according to claim 23 further including obtaining an image of the tissue using one of a CCD array and a CMOS camera disposed within the housing of the tip assembly.

25. The method according to claim 23 wherein movement of the optical component in the step of moving is assisted by an alignment member.

26. The method according to claim 23 wherein the predetermined penetration depth is less than less than 300 um.

27. The method according to claim 23 wherein the predetermined penetration depth is less than less than 100 um.

28. The method according to claim 23 wherein during the step of inserting, a hygienic sheath covers that portion of the collection optical fiber and the delivery optical fiber disposed within the cavity of the human body.

29. The method according to claim 28 wherein the housing of the tip assembly includes wherein the housing includes a finger loop, and wherein the step of inserting includes the steps of:
inserting a finger of a gloved hand into the finger loop of the housing; and
using the finger as a guide to insert the tip assembly into the cavity and within the vicinity of the tissue surface,

30. An apparatus that connects to an integrated optical fiber assembly that includes a delivery optical fiber having a light delivery end for delivering emitted broadband light and at least one collection optical fiber having a light collection end to collect interacted light, wherein the emitted broadband light illuminates one of microvasculature of tissue of a human body and tissue microarchitecture within a cavity of the human body, the apparatus comprising:
a tip assembly that releasably connects to the integrated optical fiber assembly, the tip assembly including:
a housing, wherein the housing includes a finger loop such that the tip assembly is insertable into the cavity using a finger in the finger loop;
an optical component disposed at least partially within the housing and having a tip surface that is arranged in a predetermined locational relationship, when the integrated optical fiber assembly is connected to the tip assembly, with the light collection end of the collection optical fiber and the light delivery end of the delivery optical fiber, wherein the optical component and the predetermined locational relationship are selected to provide a predetermined penetration depth of the emitted broadband light below a collection spot on a surface of the tissue when the tip surface rests on the surface of the tissue; and
a hygienic sheath having a tip end and another end with a sheath body therebetween, such that the tip end is disposed within the housing and the sheath body covers portions of the delivery optical fiber and the at least one collection optical fiber that are inserted within the cavity of the body when the integrated optical fiber assembly is connected to the tip assembly, thereby maintaining a clean environment.

31. The apparatus according to claim 30 wherein the tip assembly further includes one of a CCD array and CMOS camera mounted within the housing.

32. The apparatus according to claim 30 wherein the optical component is one of a lens and spacer, and a surface of the one of the lens and the spacer is the tip surface.

33. The apparatus according to claim 32 wherein the optical component is a lens, and wherein the optical component further includes a polarizer.

34. The apparatus according to claim 30 wherein the predetermined penetration depth is less than less than 300 um.

35. The apparatus according to claim 30 wherein the predetermined penetration depth is less than less than 100 um.

36. A obtaining an optical measurement at a tissue surface within a cavity of a human body comprising the steps of:
providing a tip assembly having an optical component with a tip surface disposed within a housing, wherein the housing of the tip assembly includes wherein the housing includes a finger loop, the optical component of the tip assembly being optically coupled to a light collection end of a collection optical fiber and a light delivery end of a delivery optical fiber;
inserting a finger of a gloved hand into the finger loop of the housing;
using the finger as a guide to insert the tip assembly into the cavity such that the tip surface rests on a collection spot of the tissue surface;
illuminating the tissue with broadband light projected through the optical component;
obtaining the optical measurement at a predetermined penetration depth below the collection spot on the surface of the tissue when the tip surface rests on the collection spot of the tissue surface.

37. The method according to claim 36 further including the steps of:
removing the tip assembly from the light collection end of the collection optical fiber and the light delivery end of a delivery optical fiber;
providing another tip assembly having another optical component with another tip surface disposed within another housing that has another finger loop;
optically coupling the another optical component of the another tip assembly to the light collection end of the collection optical fiber and the light delivery end of the delivery optical fiber; and
repeating the steps of inserting, using, illuminating and obtaining using the another tip assembly on another cavity of another human body.

38. The method according to claim 37 further including obtaining an image of the tissue using one of a CCD array and a CMOS camera disposed within the housing of the tip assembly.

39. The method according to claim 36 wherein the predetermined penetration depth is less than less than 300 um.

40. The method according to claim 36 wherein the predetermined penetration depth is less than less than 100 um.

41. The method according to claim 36 wherein during the step of using, a hygienic sheath covers that portion of the collection optical fiber and the delivery optical fiber disposed within the cavity of the human body.
